(19) United States
(12) Patent Application Publication
(10) Pub. No.: US 2006/0009725 A1
(43) Pub. Date: Jan. 12, 2006

(54) Stabilizer II

(76) Inventor: Gary W. Lampkins, Fort Washington, MD (US)

Correspondence Address: Gary W. Lampkins, 1501 Aragona Blvd., Fort Washington, MD, 20744, US

(21) Appl. No.: 11/206,830

(22) Filed: Aug. 19, 2005

Related U.S. Application Data
(63) Continuation-in-part of application No. 08/221,381, filed on Mar. 29, 1994, now Pat. No. 6,328,707.

(51) Int. Cl. A61F 5/00

(52) U.S. Cl. 602/27; 602/5; 602/23

(57) ABSTRACT

A flexible orthopedic apparatus being made a part of a shoe that will help prevent the occurrence of ankle injury due to a twist while allowing full range of motion of the foot and ankle. The apparatus accomplishes this by placing an adjustable leg attachment to the lower leg and attaching a flexible strap to the leg attachment at one end and the bottom portion of the shoe on the other. By virtue of the design, the apparatus serves as a protective extra ligament that will aid in the prevention of ankle injury.

### BACKGROUND OF THE INVENTION

The present invention relates to the stabilization and prevention of the foot and ankle rolling or turning to an injurious position while walking, running, jumping or participating in any physical activity that involves the lower extremity. The apparatus accomplishes this by attaching the lower foot to the lower leg with an adjustable, flexible extension that allows unabated foot and ankle movements.

### DESCRIPTION OF PRIOR ART

U.S. Pat. No. 4,691,698, Bremer discloses a splint with a foot plate and rigid leg support to accommodate knock knee and control proration and suppuration of the leg, knee and foot.

This design displays, however, the draw back of unabated flexibility that will allow the foot and ankle to function in its normal capacity.

U.S. Pat. No. 3,805,773 Sichau discloses a training assist brace comprising of a rigid foot and heel member attached to stirrups that extend from the foot to the thigh, designed to limit and or preclude pivotal foot and leg movements. In point, flexibility is contrary to the designs purpose and function and clearly displays its limitations of natural movement of the lower extremities.

U.S. Pat. No. 2,871,852 Miller discloses an apparatus which involves an attachment to the hip, with an elongated arm part that attaches to a foot member to prevent rotation of the foot and leg while walking. However effective, this invention by design does not incorporate individuals that are in need of flexible natural movement that is present when engaging in exercise or athletic activities.

### SUMMARY OF THE INVENTION

The main function of the invention is to provide a flexible protective shoe or attachment apparatus that will allow natural unabated movement for the wearer. Further, the invention by virtue of its design and purpose, represent a common sense approach to the limiting of ankle injury through a orthopedic athletic shoe.

### BRIEF DESCRIPTION OF THE DRAWINGS

The present invention will be clearly understood when reference is made to the detail description in conjunction to the attached drawings wherein:

FIG. 1 is a rear view of the preferred embodiments attached to the lower leg and shoe; with

FIG. 2 being a side view of the apparatus absent a shoe displaying the preferred embodiments; with

FIG. 3 being a rear view of the apparatus absent a shoe displaying the heel, ankle, and lower leg with said apparatus attached thereto; with

FIG. 4 being a rear view of the apparatus's preferred embodiments' with,

FIG. 5 being a bottom view of the apparatus attached to a specialty shoe, a cleat and the leg strap anchor, and with

FIG. 6 being a side view of the leg strap anchor.

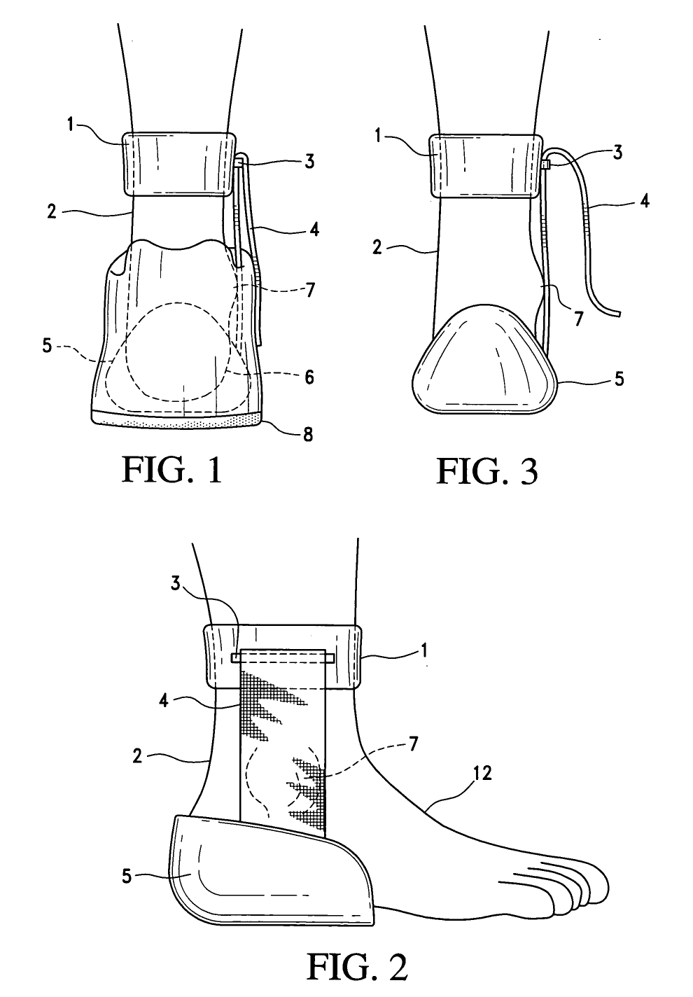

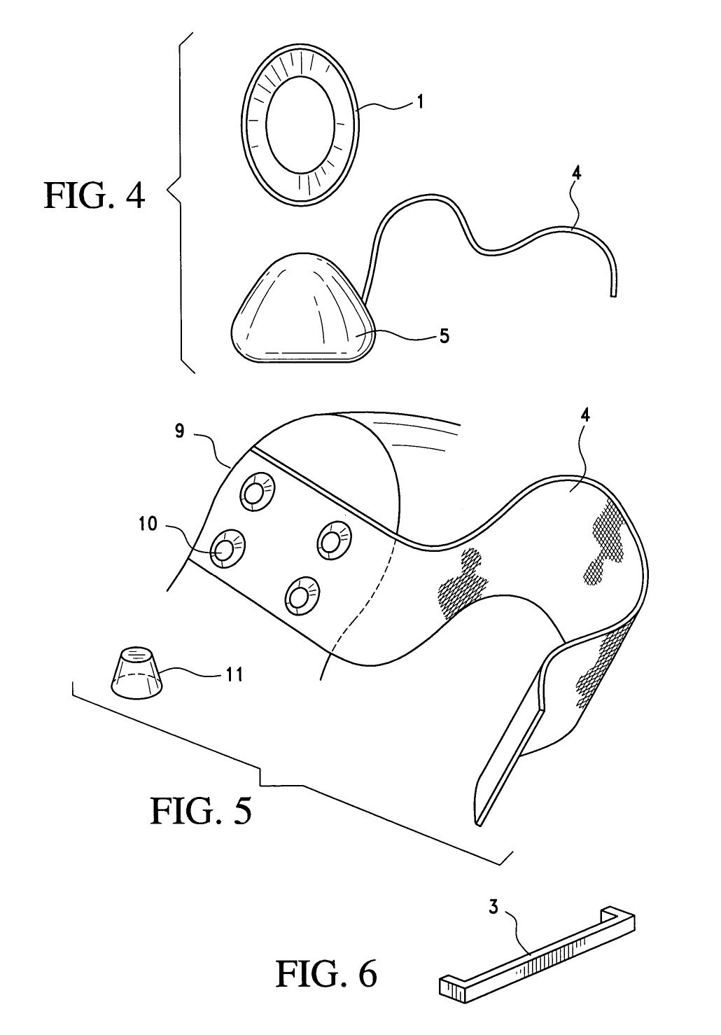

### DETAILED DESCRIPTION OF THE PREFERRED EMBODIMENT

The apparatus includes a heel cup 5 within a shoe 8 with flex strap 4 attached to heel cup 5 with flex strap 4 ascending over ankle 7 and along the lower leg 2 being inserted under the leg strap anchor 3 which is attached to the adjustable leg attachment 1; once flex strap 4 is inserted under the leg strap anchor 3, the flex strap 4 will descend along the lower leg 2 while over lapping itself and finally attaching to the shoe 8. The virtue of the flex strap 4 being attached to the heel cup 5 and attached to the adjustable leg attachment 1 by way of being inserted under the leg strap anchor 3 and finally attaching to the shoe 8 will bring functionality, flexibility and most important, stability to the invention.

I claim:

1. an orthopedic apparatus which comprises of a flexible extension attaching to the bottom portion of a shoe on one end and extending up along the lower leg, attaching to an adjustable lower leg member on the other, which will aid in the prevention of the ankle twisting to an injurious position.

2. The orthopedic apparatus of claim 1 comprising of a flexible strap which attaches to an adjustable lower leg member and continues back down the lower leg covering the ankle and reattaching to the heel cup portion of the shoe.

3. The orthopedic apparatus of claim 1 wherein the flexible strap attaches to the screws extending out of the sole of a specialty shoe such as a cleat or spike.

4. The orthopedic apparatus of claim 1 wherein the apparatus is made a part of a shoe.
